(19) United States
(12) Patent Application Publication
(10) Pub. No.: US 2014/0186945 A1
(43) Pub. Date: Jul. 3, 2014

(54) GROWTH OF CELLS

(71) Applicant: The University Court of the University of Edinburg, Edinburgh (UK)

(72) Inventors: Mark Bradley, Edinburgh (GB); Paul Alexandre De Sousa, Midlothian (GB); Rong Zhang, Edinburgh (GB); Heidi Katharina Mjoseng, Edinburgh (GB)

(21) Appl. No.: 14/125,812

(22) PCT Filed: Jun. 14, 2012
(86) PCT No.: PCT/GB2012/000517

§ 371 Date: Mar. 3, 2014

(30) Foreign Application Priority Data
Jun. 14, 2011 (GB) 1110042.7

(51) Int. Cl. C12N 5/0735

(52) U.S. Cl. CPC C12N 5/0606
USPC 435/352; 435/363; 435/366; 435/378; 435/396

(57) ABSTRACT

The present invention relates to the use of certain polymers as a substrate for stem cell, such as pluripotent stem cell growth and/or culture. The present invention also relates to articles such as tissue culture materials and cell culture devices comprising at least one polymer hydrogel as described herein.

### FIELD OF THE INVENTION

The present invention relates to the use of certain polymers as a substrate for stem cell, such as pluripotent stem cell growth and/or culture. The present invention also relates to articles such as tissue culture materials and cell culture devices comprising at least one polymer hydrogel as described herein.

### BACKGROUND OF THE INVENTION

Reliable culture systems for human pluripotent stem cells, such as those obtained from embryonic or adult tissue, suitable for application in biological and clinical research and therapies requires chemical definition of supportive, cost-effective reagents.^{1,2,3}

Several feeder-independent and defined media formulations with the capacity to maintain both an undifferentiated phenotype and cellular differentiation potential have been described,^{4,5,6} and exemplified using human embryonic stem cells (human ESCs) which constitutes the gold standard for pluripotent stem cells including those induced by gene transfection and/or small molecules or isolated from adult tissues. With the exception of a few carrier-free suspension culture systems for human ESCs,^{7,8} carrier-dependent⁹ or adherent feeder-free culture systems rely on recombinant or purified matrices to support attachment and growth. These systems typically include a range of recombinant proteins, lipids, nutrients and small molecules that affect the intracellular signalling pathways involved in cell death, growth and differentiation. These substrates include commercially available products such as Matrigel (MG) and Cell Start, as well as extracellular matrix proteins such as laminin,¹⁰ fibronectin,¹¹ vitronectin¹² or combinations thereof.⁵ Recently, screening approaches have identified polymer and peptide-polymer substrates exhibiting an ability to sustain a pluripotent cell phenotype.^{13,14,15,16} Significant caveats are associated with these advances, such as the need to select for pluripotency at passage, e.g. by manual colony picking,¹⁴ the need for enzymatic dissociation,^{10,16} or the requirement for supplementation with undefined reagents such as serum for adhesion.¹⁶

In contrast, thermally responsive hydrogels which swell and become increasingly rigid upon temperature reduction are particularly suited for stem cell culture and could help to overcome some of the shortfalls of established systems.¹⁷ This phenomenon would also address issues that prevent stem cells and pluripotent stem cells from being employed in a clinical setting, and would allow large-scale culturing, easy detachment and re-seeding without the use of enzymes or complex biological matrices.^{7,18,19}

### SUMMARY OF THE INVENTION

The present invention is based on the identification by the inventors of certain polymer hydrogels which are able to support stem cells, such as pluripotent stem cell attachment and/or growth and that are able to allow detachment of the stem cells/pluripotent stem cells upon a lowering of its temperature (referred to herein as thermal treatment). Advantageously the present invention provides polymer hydrogels which permit log-term (e.g. >2-6 months) maintenance of stem cells/pluripotent stem cells, such as human embryonic stem cells (hESC), whilst at the same time allowing reagent free dissociation of the cells in response to a reduction in temperature.

In a first aspect of the present invention there is provided a method of culturing stem cells, such as pluripotent stem cells on a substrate comprising a polymer hydrogel which may be a polyacrylate or a polyacrylamide wherein the stem cells/pluripotent stem cells may be released from the substrate upon thermal treatment.

Preferably the polymer comprises a random structure according to the following composition:

Xₙ; Y_{n′}; Z_{n″}

wherein X and Y represent monomers and Z represents a cross-linking agent; and
wherein X may be selected from hydroxypropyl methacrylate (HPMA), [2-(acryloyloxy)ethyl]trimethylammonium chloride (AEtMA-Cl), 2-(dimethylamino)ethyl methacrylate (DMAEMA); and
wherein Y may be selected from 2-(diethylamino)ethyl acrylate (DEAEA), N-(1,1-dimethyl-3-oxobutyl)acrylamide (DMOBAA), N-isopropylacrylamide (NIPAA), and N,N-diethylacrylamide (DEAA); and
wherein Z may be selected from N,N′-methylenebisacrylamide (MBA), ethylene glycol diacrylate, tetra(ethylene glycol) diacrylate, glycerol dimethacrylate, poly(ethylene glycol) diacrylate and (O,O-Bis(3-aminopropyl)polyethylene glycol) diacrylamide; and wherein n and n′ respectively represent the molar ratio in percent of monomers X and Y relative to each other and may be from 10%-90%; 90-10%; and
wherein n″ represents the molar ratio in percent of Z relative to X and Y combined and may be from 1%-20%.

The skilled person would understand that the monomers X and Y and the cross-linking agent Z will be present in the polymer hydrogel structure as their respective residues, after the polymerisation reaction has taken place.

In use, thermodetachment of stem cells/pluripotent stem cells allows, for example, cell passaging. For example, thermodetachment allows stem cells/pluripotent stem cells to be transferred from a substrate comprising at least one polymer hydrogel of the present invention to another substrate or into media. In terms of thermodetachment, preferably at least 50%, 60%, 70%, 80% or 90% of the cells which are initially attached to the hydrogel, may be released upon thermo-modulation.

In use, the growth of stem cells/pluripotent stem cells is supported by polymer hydrogels of the present invention. In use, the polymer hydrogels of the present invention demonstrate stable maintenance of undifferentiated phenotypes of stem cells/pluripotent stem cells. In use, polymer hydrogels of the present invention carrying stem cells/pluripotent stem cells may be used to transfer the cells to a location in vivo and thermal treatment may allow the release of stem cells/pluripotent stem cells from the polymer hydrogel into the subject. In use, polymer hydrogels of the present invention may be deployed in vivo wherein stem cells/pluripotent stem cells present in the subject may attach to the polymer hydrogel and thus allow the harvesting of pluripotent stem cells from the subject. In use, polymer hydrogels of the present invention may be deployed to attach cells to facilitate gene transfection or induction of differentiation or death using other reagents.

Preferably n and n′ may be from 20-80%; 80-20%. For example n and n′ may be from 25-75%; 75-25%. Especially n and n′ may be from 30-70%; 70-30%.

Preferably n″ may be from 2-10%. Especially, n″ may be from 4-8%. In particular, n″ may be approximately 6.5%.

In preferred embodiments, X may be selected from HPMA and AEtMA-Cl. In preferred embodiments Y may be DEAEA.

Preferably n and n′ may be from 20-80%; 80-20%. For example n and n′ may be from 25-75%; 75-25%. Especially n and n′ may be from 30-70%; 70-30%.

Preferably n″ may be from 2-10%. Especially, n″ may be from 4-8%. In particular, n″ may be approximately 6.5%.

Preferably the polymer hydrogel may be a random co-polymer. In a preferred embodiment, the polymer hydrogel may comprise segments of monomeric units derived from X and Y wherein the monomeric units are of random length. Monomeric units of X and Y may comprise one monomer up to any number of monomers. The structure of the polymer hydrogel may conform to any random combination of X and Y according to its composition. The cross-linking agent Z may randomly cross-link different polymeric segments.

In one embodiment, preferred polymers comprise the following composition:

AEtMA-Clₙ; DEAEA_{n′}; MBA_{n″}

wherein n and n′ may be from 30-70%; 70-30%; or

wherein n and n′ may be from 33.3-66.6%; 66.6-33.3%; or

wherein n and n′ may be 33.3% and 66.6% respectively; or

wherein n and n′ may be each 50%; or

wherein n and n′ may be 66.6% and 33.3% respectively.

Stated otherwise, the ratio of n to n′ may be 1:3, 1:1 or 3:1. In a preferred embodiment the ratio of n to n′ is 3:1.

n″ may be from 4-8%. For example, n″ may be approximately 6.5%.

In one embodiment, preferred polymers comprise the following composition:

HPMAₙ; DEAEA_{n′}; MBA_{n″}

wherein n and n′ may be from 30-70%; 70-30%; or

wherein n and n′ may be from 33.3-66.6%; 66.6-33.3%; or

wherein n and n′ may be 33.3% and 66.6% respectively; or

wherein n and n′ may be each 50%; or

wherein n and n′ may be 66.6% and 33.3% respectively.

Stated otherwise, the ratio of n to n′ may be 1:3, 1:1 or 3:1.

n″ may be from 4-8%. For example, n″ may be approximately 6.5%.

Illustrations of the monomeric residues X and Y in embodiments of the present invention are shown in formulae I and II below:

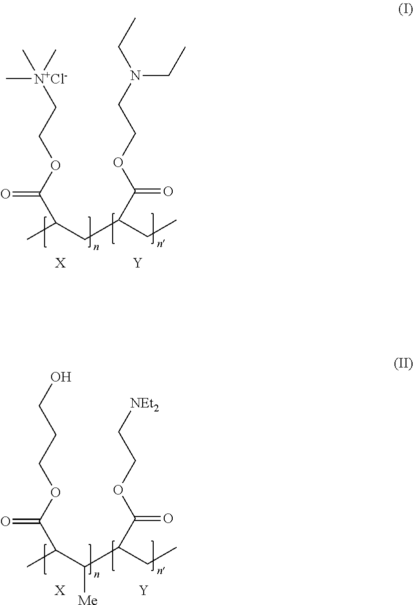

The present invention provides a method comprising culturing stem cells, such as pluripotent stem cells on a polymer hydrogel. The method may comprise seeding stem cells/pluripotent stem cells on a substrate comprising a polymer hydrogel of the present invention and then cultivating and/or growing the stem cells/pluripotent stem cells on the substrate. The method comprises removing stem cells/pluripotent stem cells from the polymer hydrogel under mild thermal conditions. The removed stem cells/pluripotent stem cells may then be re-seeded on another tissue culture substrate which may comprise at least one polymer hydrogel of the present invention or another suitable substrate

One of skill in the art will be familiar with the term “stem cells” which may take the form of “adult”, “embryonic” and/or induced pluripotent stem cells (iPS). Furthermore, the term “stem cells” may encompass mammalian and in particular, human, stem cells. Nevertheless, non-human mammalian stem cells, including those derived from primates, ungulates, ruminants and/or rodents are included within the term “stem cells” and the uses, methods and compositions provided by this invention may find application in the culture or maintenance of stem cells derived from sheep, pigs, cattle, goats, horses, rats and mice. The skilled reader is directed to a review refererenced herein (Ng, H-H.; Azim Surani, M. The transcriptional and signalling networks of pluripotency, Nature Cell Biology, 13 (5), 490-496 (2011)) which provides a discussion in the area of pluripotency and the types of cell that this term encompasses.

More generally, the term “stem cells” may be taken to refer to any cell which is able to self renew and indefinitely divide—cells of this type may occasionally be described as “immortal”. In addition, when cultured under suitable conditions and/or contacted with, or exposed to, particular compounds and/or conditions, stem cells may differentiate into any one of the specialised cell types which form embryonic and/or adult tissues.

Embryonic stem cells (ESC), for example, mammalian and/or rodent/human embryonic stern cells are derived from early stage embryos and in particular from the inner cell mass of the developing morula or blastocyst. Embryonic stem cells derived from embryos in the stages immediately following conception (and for a short time thereafter) may be totipotent and thus capable of generating a complete viable organism as well as differentiating into any given specialised cell type. Embryonic stem cells derived from later stage embryos (i.e. from the late epiblast of a developing blastocyst) although pluripotent and capable of differentiating to any specialised cell type, are not capable of generating a complete viable organism. It is to be understood that this invention relates to methods and compositions for maintaining mammalian totipotent and/or pluripotent stem cells in culture. In certain embodiments the embryonic stem cells are not totipotent.

Alternatively, and in order to avoid having to use embryonic material, stem cells may be obtained from established ESC lines A number of human ESC lines may be found in the NIH Human Embryonic Stem Cell Registry, to which the skilled reader is directed (NIH, Bethesda, Md., USA). Representative examples of ESC lines include, but are not limited to RH1, RCM1 and H9 cell lines.

Additionally or alternatively, the hESCs and cell lines may be obtained from an embryo without destruction of the embryo, as described, for example, in Chung et al (Cell Stem Cell, vol 2, issue 2, 113-117, 2008).

The term “stem cells” may also encompass cells known as induced pluripotent stem cells (iPS). These are adult somatic cells which have been modified to express certain transcription regulators and as a consequence become pluripotent and thus capable of differentiating to any other specialised cell type. As such, iPS cells represent a possible source of cells which may be cultured and/or maintained with the methods and/or compositions described herein. iPS cells may be derived from primary human cells (iPSC) or from adult tissue (e.g. adipose derived stem cells).

It should be understood that key markers of undifferentiated stem cells may include, for example, levels (for example high levels) of nuclear Oct4 and/or Nanog protein, Rex1 expression (a transcription factor that is highly expressed in the early epiblast and down-regulated in late epiblast derivatives). Furthermore, the absence of, for example FGF5 expression—a marker of the late-epiblast, may be used to confirm the primitive epiblast status of stem cells cultured in accordance with the methods described herein. A list of suitable markers is provided in Buehr et al., 2008—the contents of which is incorporated herein by reference.

The term “stem cells” may also be taken to refer to the pluripotent cells derived from the three primary germ layers (ectoderm, mesoderm and endoderm) which develop during the process of gastrulation.

One of skill in this field will appreciate that cells derived from these layers may express one or more markers which may be used as a means of identification. By way of example, ectoderm germ layer may express markers, including, for example, Otx2, Nestin, TP63/TP73L, beta-III Tubulin, SHH, and PAX6. Ectoderm has the potential to form cell types such as neurons and early neuronal lineage markers include ACE, ALCAM, CD90/Thy1, GAD1/GAD67, Glut1, MAP2, NCAM-L1, Nectin-2/CD112, NeuroD1, NF-L, NF-M, ROBO3, gamma-Secretase, alpha-Secretase, beta-Secretase, beta-III tubulin (Tuj 1), Tyrosine Hydroxylase. Neural stem cell markers include ABCG2, CXCR4, FGF R4, Frizzled-9, Musashi-1, Nestin, Noggin, Nucleostemin, Prominin 2, SOX2, Vimentin. Markers of early endodermal cells include, for example, FABP1, FABP2, GATA-4, HNF-3 beta (collectively referred to as definitive endodermal stem cells markers) as well as those markers for primitive endoderm such as alpha-Fetoprotein (AFP), beta-Catenin, GATA-4, SOX17 and SOX7.

The uses methods and compositions described herein may also be used to maintain mesenchymal stem cells or cultures or populations thereof. One of skill in this field will appreciate that mesenchymal stem cells are multipotent in that they have the capacity to form a number of more specialised cell lineages including, for example chondrocytes, osteocytes, adipocytes, cardiomyocytes, myoblasts and cells of the connective tissue such as, for example, fibroblasts. In addition, mesenchymal stem cells may be characterised by the fact that they express a number of specific markers including, CD71, CD90, GATA6, Nodal, BMP-2. Furthermore, mesenchymal stem cells may express one or more of the markers selected from the group consisting of BMPR-1A/ALK-3; BMPR-IB/ALK-6; BMPR-II; Endoglin/CD105; Nucleostemin; Sca-I; SCF R/c-kit; STRO-1 and VCAM-1.

It should be understood that the term “maintaining” stem cells refers to the act of sustaining stem cells/pluripotent stem cells for prolonged periods of time in an undifferentiated state, retaining the ability to differentiate to adult cell lineages. Typically the polymers of the present invention allow the cells to remain in an undifferentiated state and retain multi/pluripotency for at least 1 month, preferably greater than 2, 3, 4, 5, 6 months, or even longer.

In other words, maintained stem cells/pluripotent stem cells are exposed to or cultured in or with certain conditions or factors which ensure the cells remain proliferative, self renew, do not differentiate and/or commit to any particular linage and substantially retain the morphological and phenotypic features characteristic of stem cells. The polymer hydrogels of the present invention may allow stem cells/pluripotent stem cells to maintain the capacity and/or viability to differentiate into different cell lines. Stem cells/pluripotent stem cells cultured on polymer hydrogels of the present invention may show stable maintenance of the undifferentiated phenotype. Pluripotent stem cells cultured on polymer hydrogels of the present invention maintain the viability to differentiate down endoderm, mesoderm and ectoderm cell lines. In addition, the method and compositions described herein may be used to maintain stem cells for prolonged periods of time.

“Passaging” as used herein refers to a technique of transferring a small proportion of cells to a new substrate or vessel. Passaging enables cells to be maintained alive and growing under cultured conditions for extended periods of time. One of skill in this field will be familiar with the term passage and will know that under standard culture conditions stem cells may only be maintained for finite and often short periods of time before differentiating or dying. The present invention provides methods and compositions which may be used to maintain stem cells/pluripotent stem cells through numerous different passages. Polymer hydrogels of the present invention may be used to culture stem cells/pluripotent stem cells indefinitely. Typically, polymer hydrogels of the present invention allow stem cells/pluripotent stem cells to be passaged over 20 times. Stem cells passaged on polymer hydrogels of the present invention maintain the capability of differentiating down different cell lines. For example, pluripotent stem cells passaged on polymer hydrogels of the present invention maintain the capability of differentiating down endoderm, mesoderm and ectoderm cell lines

“Culturing” as used herein refers to the growth, maintenance, storage and passaging of cells. Cell culture techniques are well understood and often involve contacting cells with particular media to promote growth. In the present case, stem cells contacted with or exposed to polymer hydrogels of the present invention during culture may continue to grow and proliferate, maintaining their undifferentiated phenotype throughout the culture period or for a prolonged period of time.

The base-substrate may be a solid or semi-solid substrate. Suitable examples may include base-substrates comprising, for example, glass, plastic, nitrocellulose or agarose. In one embodiment, the base-substrate may take the form of a glass or plastic plate or slide. In other embodiments, the base-substrate may be a glass or plastic multi-well plate such as, for example a micro-titre plate. In one embodiment the base-substrate may take the form of a tissue culture flask, roller flasks or multi-well plate. The base-substrate may be coated with the polymer hydrogel. The base-substrate may be coated with a layer or several layers of the polymer hydrogel. The polymer hydrogel may be incorporated into the main body of the substrate.

Thermal treatment may comprise subjecting the polymer hydrogel to a particular temperature regime. Thermal treatment may comprise cooling the polymer hydrogel to a lower temperature than the initial temperature. The initial temperature may be the incubation temperature of the pluripotent stem cells. For example, the initial temperature may be between 30-39° C., and typically 37° C. Thermal treatment comprises cooling the polymer hydrogel to a particular temperature and then maintaining the polymer hydrogel at that particular temperature for a period of time. Thermal treatment may comprise cooling the polymer hydrogel to a temperature between 5 and 30° C. In a preferred embodiment, thermal treatment may comprise cooling the polymer hydrogel to between 10 and 18° C. The polymer may be held at a particular temperature for a particular length of time, for example from 1 minute up to 12 h. Preferably the polymer hydrogel is maintained at the desired temperature for 30 minutes. In a preferred embodiment, thermal treatment comprises lowering the temperature of the polymer hydrogel from approximately 37° C. to approximately 15° C. for approximately 30 minutes.

Thermodetachment as used herein refers to the removal of cells from a substrate after a thermal stimulus is applied. In particular, thermodetachment may refer to the process of removing pluripotent stem cells from a substrate comprising a polymer hydrogel of the present invention after cooling to a particular temperature.

Thermal treatment of the polymer hydrogels of the present invention has an effect on the structural and physical features of the polymers. The effect of thermal treatment of the polymer hydrogel properties may be reversible. Polymer hydrogels of the present invention may be referred to as thermoresponsive. Polymer hydrogels of the present invention may swell upon cooling. Polymer hydrogels of the present invention may swell up to 30% upon cooling. In a preferred embodiment, polymer hydrogels of the present invention may swell between 4-15% upon cooling. Polymer hydrogels of the present invention may become more rigid upon temperature reduction. The storage modulus of polymer hydrogels of the present invention may increase with temperature. The effect on the storage modulus may be reversible. For example, the storage modulus of the polymer hydrogel may increase with temperature and then be decreased upon cooling. The storage modulus of polymer hydrogels of the present invention may be between 10 and 10000 Pa. In a preferred embodiment, the storage modulus may be between 100 Pa and 5000 Pa.

The polymers of the present invention find particular application in cell culture products designed to facilitate the culture of stem cells/pluripotent stem cells. The polymers may be used for culturing of stem cells/pluripotent stem cells in vitro. The polymers may form part of a tissue culture substrate. The polymers may be used to coat the base-surface of tissue culture substrates such as the base-surface of microtitre plates, cell culture flasks, roller flasks and the like. Typically only a base-surface which comes into contact with cells need be coated.

Thus, according to a second aspect of the present invention, there is provided a cell culture device or apparatus for use in the culture of stem cells, such as pluripotent stem cells comprising at least one polymer hydrogel as described here in and a base-substrate.

The tissue culture apparatus may be pre-seeded with stem cells/pluripotent stem cells or the apparatus may be ‘naked’ i.e. there may be no pluripotent stem cells present.

The tissue culture apparatus may comprise a growth medium to support cell culture. The tissue culture apparatus may comprise nutrients, antibiotics and other such additives to support cell culture.

According to a third aspect of the present invention, there is provided a polymer hydrogel which comprises a random structure according to the following composition:

Xₙ; Y_{n′}; Z_{n″}

wherein X and Y represent monomers and Z represents a cross-linking agent; and

wherein X may be selected from hydroxypropyl methacrylate (HPMA), [2-(acryloyloxy)ethyl]trimethylammonium chloride (AEtMA-Cl), 2-(dimethylamino)ethyl methacrylate (DMAEMA); and

wherein Y may be selected from 2-(diethylamino)ethyl acrylate (DEAEA), N-(1,1-dimethyl-3-oxobutyl)acrylamide (DMOBAA), N-isopropylacrylamide (NIPAA), and N,N-diethylacrylamide (DEAA); and

wherein Z may be selected from N,N′-methylenebisacrylamide (MBA) ethylene glycol diacrylate, tetra(ethylene glycol) diacrylate, glycerol dimethacrylate, poly(ethylene glycol) diacrylate and (O,O-Bis(3-aminopropyl)polyethylene glycol) diacrylamide; and

wherein n and n′ respectively represent the molar ratio in percent of monomers X and Y relative to each other and may be from 10%-90%; 90-10%; and

wherein n″ represents the molar ratio in percent of Z relative to X and Y combined and may be from 1%-20%.

The skilled person would understand that the monomers X and Y and the cross-linking agent Z will be present in the polymer hydrogel structure as their respective residues, after the polymerisation reaction has taken place.

Preferably n and n′ may be from 20-80%; 80-20%. For example n and n′ may be from 25-75%; 75-25%. Especially n and n′ may be from 30-70%; 70-30%.

Preferably n″ may be from 2-10%. Especially, n″ may be from 4-8%. In particular, n″ may be approximately 6.5%.

In preferred embodiments, X may be selected from HPMA and AEtMA-Cl. In preferred embodiments Y may be DEAEA.

Preferably n and n′ may be from 20-80%; 80-20%. For example n and n′ may be from 25-75%; 75-25%. Especially n and n′ may be from 30-70%; 70-30%.

Preferably n″ may be from 2-10%. Especially, n″ may be from 4-8%. In particular, n″ may be approximately 6.5%.

Preferably the polymer hydrogel may be a random co-polymer. In a preferred embodiment, the polymer hydrogel may comprise segments of monomeric units derived from X and Y wherein the monomeric units are of random length. Monomeric units of X and Y may comprise one monomer up to any number of monomers. The structure of the polymer hydrogel may be any sequential combination of X and Y. The cross-linking agent Z may cross-link different polymeric segments at many different points.

In one embodiment, preferred polymers comprise the following composition:

AEtMA-Clₙ; DEAEA_{n′}; MBA_{n″}

wherein n and n′ may be from 30-70%; 70-30%; or

wherein n and n′ may be from 33.3-66.6%; 66.6-33.3%; or

wherein n and n′ may be 33.3% and 66.6% respectively; or

wherein n and n′ may be each 50%; or

wherein n and n′ may be 66.6% and 33.3% respectively.

Stated otherwise, the ratio of n to n′ may be 1:3, 1:1 or 3:1. Preferably the ratio of n to n′ is 3:1.

n″ may be from 4-8%. For example, n″ may be approximately 6.5%.

In one embodiment, preferred polymers comprise the following composition:

HPMAₙ; DEAEA_{n′}; MBA_{n″}

wherein n and n′ may be from 30-70%; 70-30%; or

wherein n and n′ may be from 33.3-66.6%; 66.6-33.3%; or

wherein n and n′ may be 33.3% and 66.6% respectively; or

wherein n and n′ may be each 50%; or

wherein n and n′ may be 66.6% and 33.3% respectively.

Stated otherwise, the ratio of n to n′ may be 1:3, 1:1 or 3:1.

n″ may be from 4-8%. For example, n″ may be approximately 6.5%.

Illustrations of the monomeric residues X and Y in embodiments of the present invention are shown in formulae I and II below:

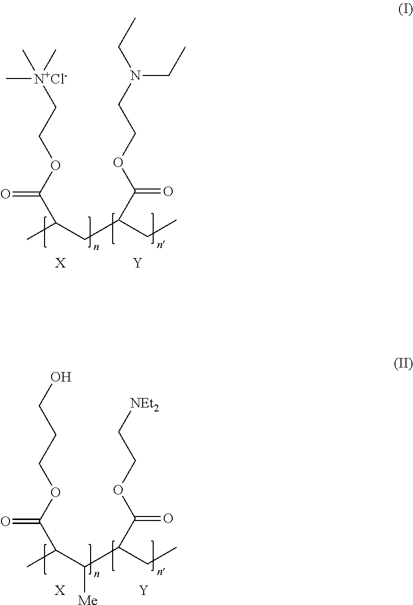

### DETAILED DESCRIPTION OF THE INVENTION

The present invention will now be further described with reference to the following figures which show:

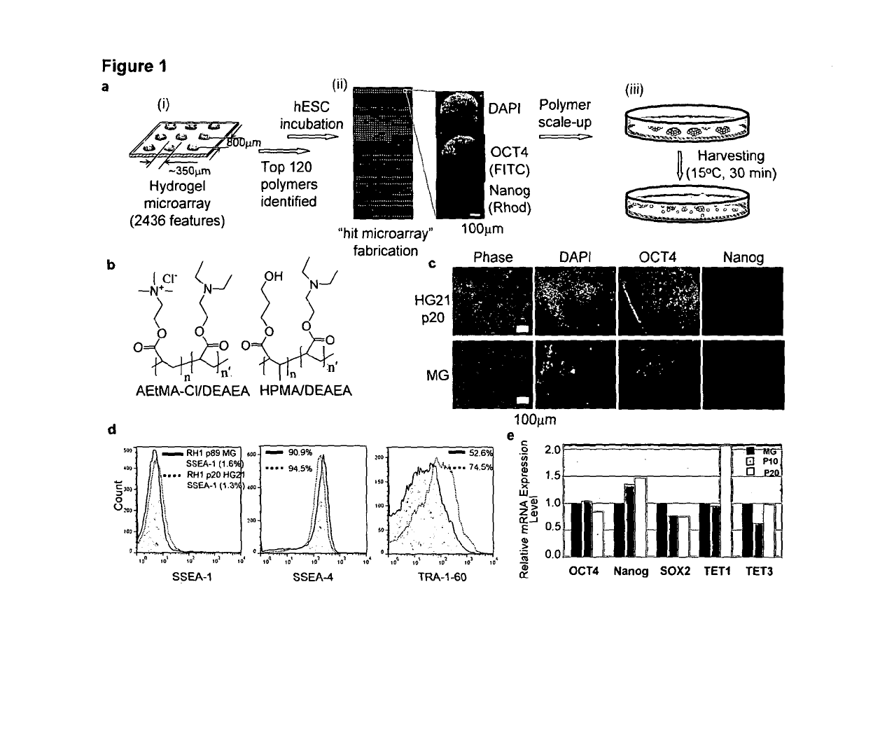

FIG. 1: (a) High throughput screening of polymer hydrogels for long-term support of hESC growth, control of differentiation and enzyme free passaging.

(i). Hydrogel microarrays with 609 different polymers (in quadruplicate) were fabricated by in situ polymerisation on a glass microscope slide. The microarrays were treated with human ESCs for 24 hours before fixing and staining with DAPI.

(ii). Mosaic image of the top 120 hydrogels (with nine copies of each) re-synthesized and cultured with human ESCs for 2, 4 and 7 days before fixing and immunostaining for OCT4/Nanog. The expansion shows a single polymer feature with human ESCs cultured for 7 days (upper=DAPI, middle=OCT4 and lower=Nanog).

(iii). Four candidates (showing good cellular binding and thermal-release properties and the highest number of OCT4/Nanog positive cells) were synthesized on glass coverslips. The cells were cultured for over 20 passages using, at each step, temperature triggered detachment. Briefly, once 70%-80% confluency was reached, cells were incubated at 15±3° C. for 30 minutes, most of the cells were detached and could be broken down to smaller cellular aggregates by gentle pipetting. Then the cells were plated onto fresh polymer hydrogel coverslips.

(b) Structure of the hydrogels HG9 (HPMA/DEAEA (n=1, m=3) and HG19, 20 and 21 (AEtMA-Cl/DEAEA (n=3, n′=1; n=1, n′=1 and n=1, n′=3).

(c) Images of cell colonies showing expression of hESC markers OCT4 and Nanog after culture on hydrogel HG21 after 20 passages.

(d) Flow cytometry analysis of human ESCs thermo-detached from HG21 and enzymatically harvested from MG respectively.

(e). qRT-PCR comparison of expression levels of the pluripotency markers OCT4, Nanog, SOX2, TET1 and TET3 at passages 10 and 20 of culture of cells grown on HG21, normalised to the “housekeeping gene” GAPDH (shown as a ratio with respect to RH1 cells cultured on MG)

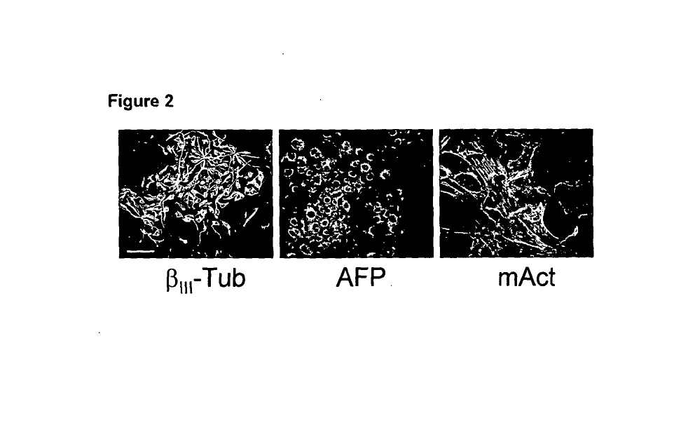

FIG. 2: Fluorescent images of in vitro differentiated RH1 human ESCs harvested from HG21 after 20 passages cultured in mTeSR1 medium and subjected to a standard embroid body mediated differentiation protocol. Images depict immunoreactivity for β-III tubulin (ectoderm), α-fetoprotein (endoderm) and smooth muscle actin (mesoderm) demonstrating the pluripotent state of RH1 cells.

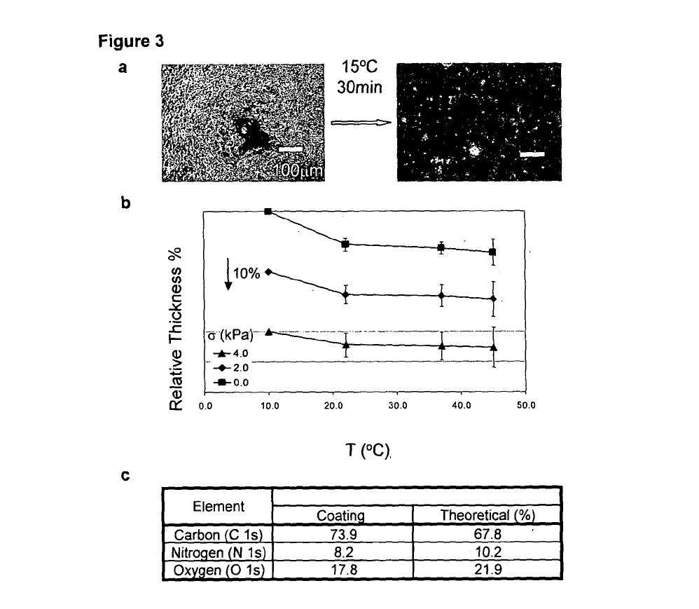

FIG. 3: Analysis of HG21.

(a) Thermo-detachment of RH1 from HG21 by incubation of the cells at 15° C. for 30 minutes. (b) Thickness change of bulk HG21 (in PBS) with temperature under different compressive strengths. Relative thickness measurements were determined in the range of 1.6-11.3 kPa (only two lines shown). Extrapolation of this data was used to give the properties of the hydrogel under physiological conditions (±SD, n=3). (c) X-ray photoelectron spectroscopy (XPS) analysis of dried HG21 showing that the polymerisation proceeded according to the starting monomer ratios.

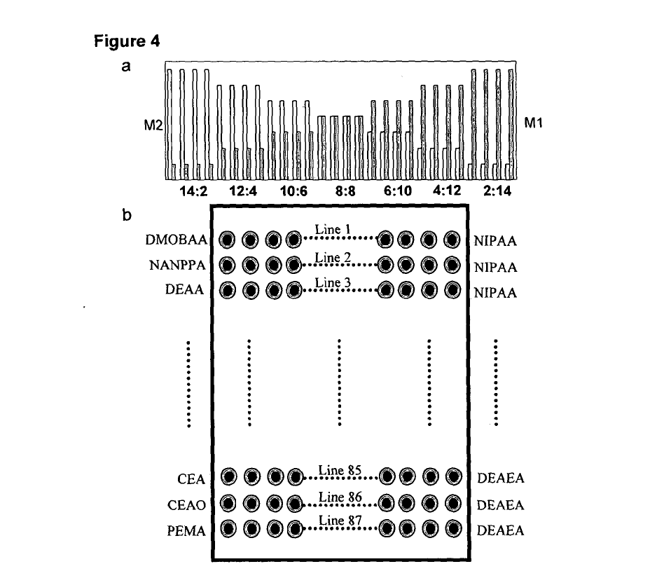

FIG. 4: a) Ratio of drops of monomer printed at each position. b) The pattern of the printed hydrogel microarray. The pitch between polymer spots was 0.8 mm. The 6 monomers in first column in Table 1 are the main monomers. During printing, each forms two-monomer combinations with all other monomers respectively. Briefly, each line along the width direction of a slide, two monomers overprinted at each spots, was printed with 7 ratios in drops, which are 14/2, 12/4, 10/6, 8/8, 6/10, 4/12 and 2/14 respectively.

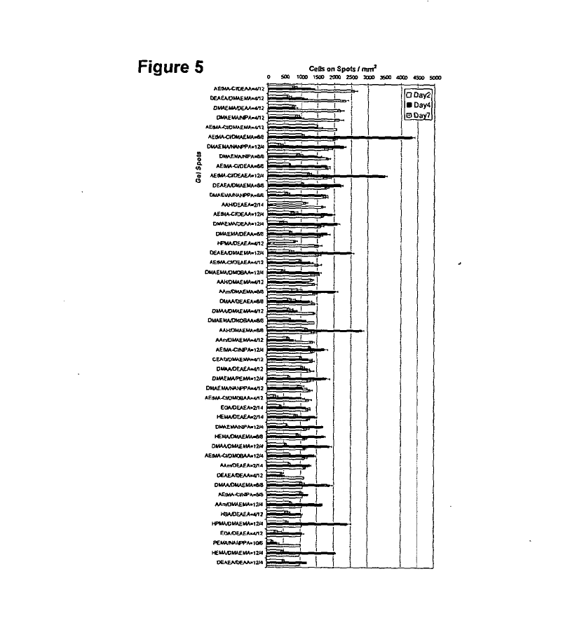

FIG. 5: Average RCM1 hESC binding on the top 50 hydrogel polymers after 2, 4 and 7 days culture in medium with bFGF.

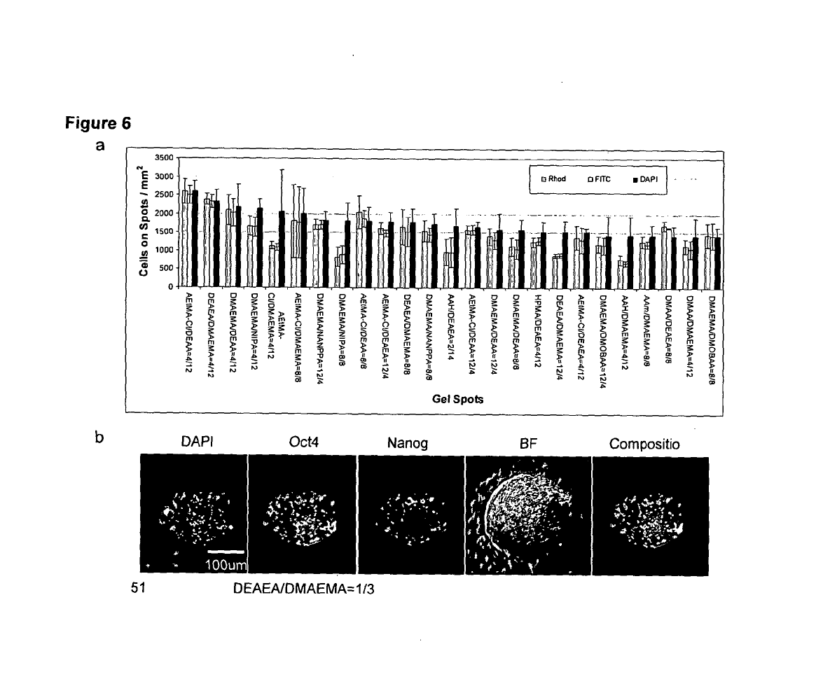

FIG. 6: (a) Comparison of the number of cells stained with Nanog (Rhod), OCT4 (FITC) and DAPI on top 25 hydrogels after 7 days culture in mTeSR with bFGF. (b) Fluorescent and bright field images of human ESCs bound on polymer spots after 7 days culture stained with Hoechst 33342 (DAPI), OCT4 (FITC) and Nanog (Rhodamine). Compositions are the merging images of three colours. Culture medium was mTeSR1 with bFGF. Small colonies were formed on the spots.

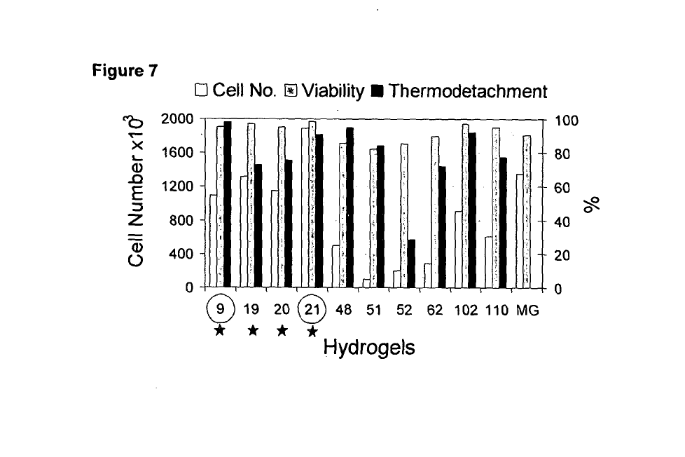

FIG. 7: hESC growth on top 10 hydrogels and the viability after thermodetachment treated under 15° C. for 30 min.

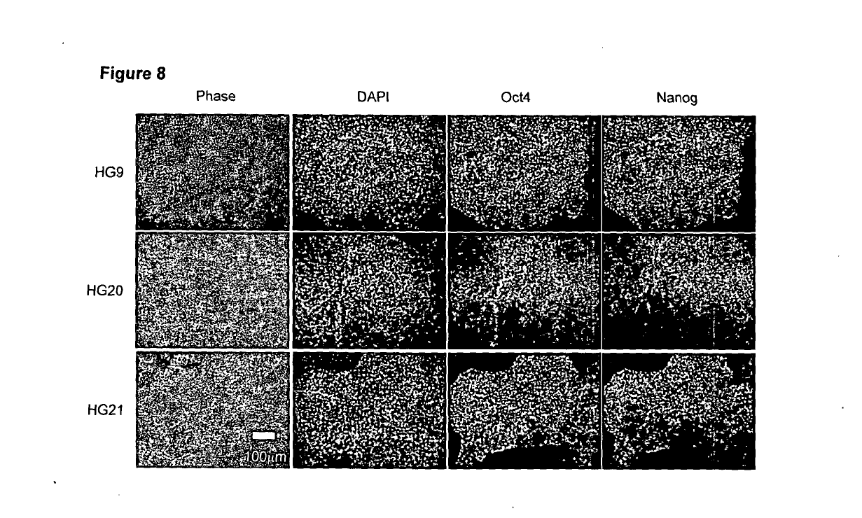

FIG. 8: Markers/Morphology of RH1 hESC on 3 hydrogels at p10.

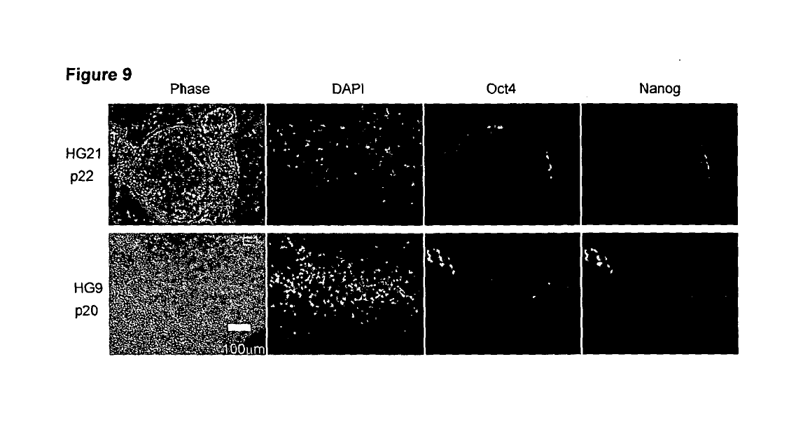

FIG. 9: Immunostaining of RCM1 hESC at p20 harvested from HG9 and HG21.

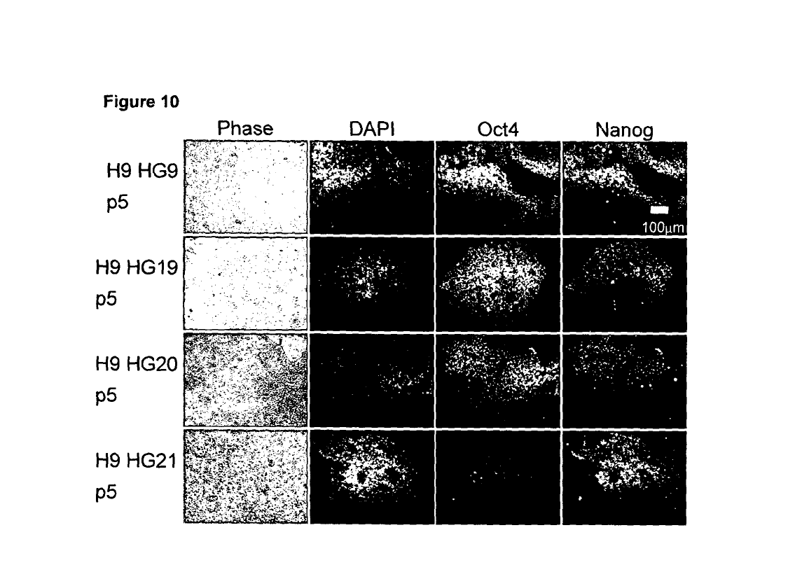

FIG. 10: Immunostaining of H9 hESC at p5 harvested from HG9, HG19, HG20 and HG21.

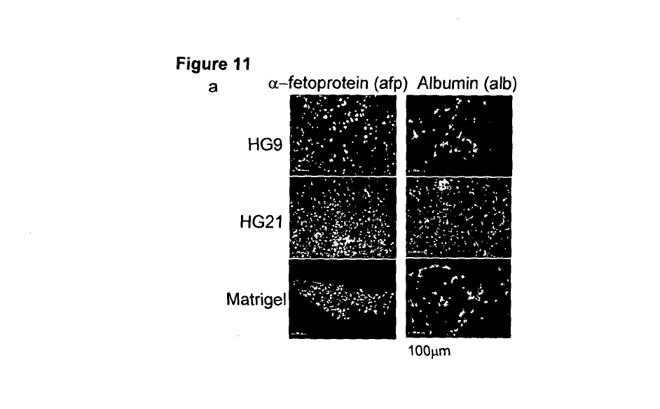

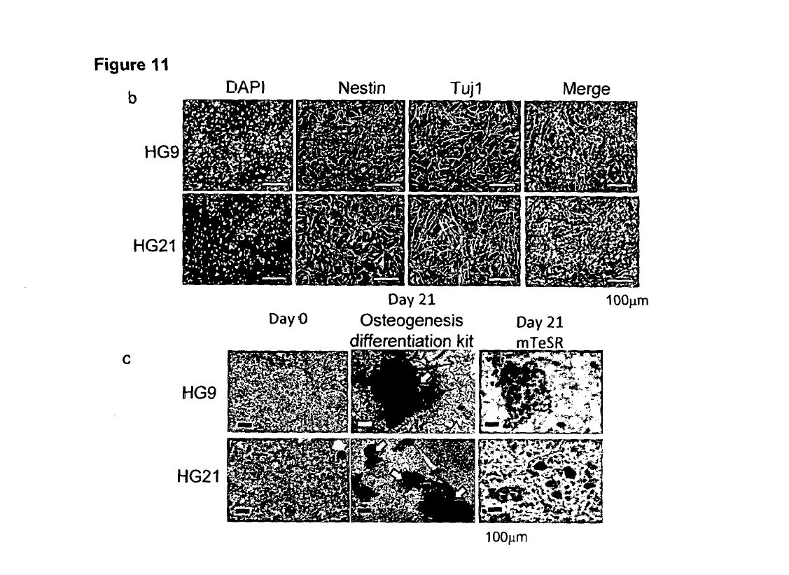

FIG. 11: Immunostaining of differentiated cells from RCM1 hESC after 20 passages on polymer hydrogels revealed expression of markers for the three embryonic cell layers: (a) Endoderm, AFP and ALB, (b) Ectoderm, nestin and Tuj1 and (c) Mesoderm, Von Kossa-stained calcium phosphate.

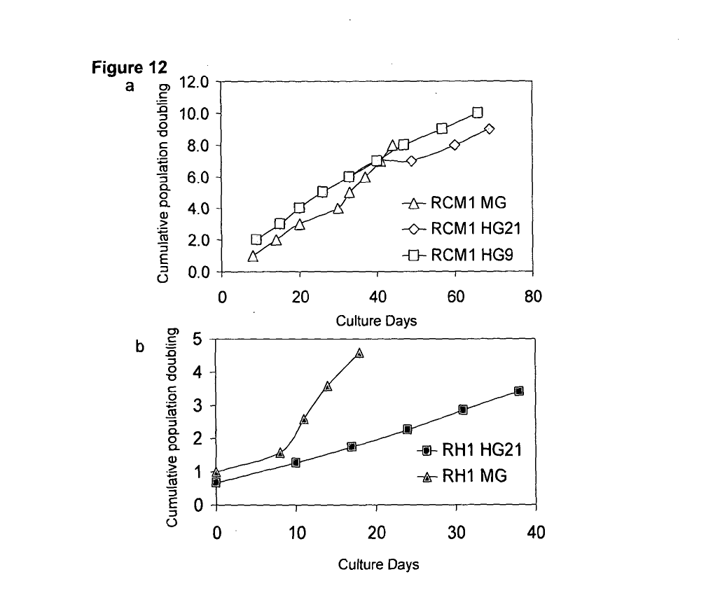

FIG. 12: Cumulative population of human ESCs on hydrogels compared with cells on Matrigel. (a) RCM1 cells on MG, HG21 and HG9; (b) RH1 cells on MG and HG21.

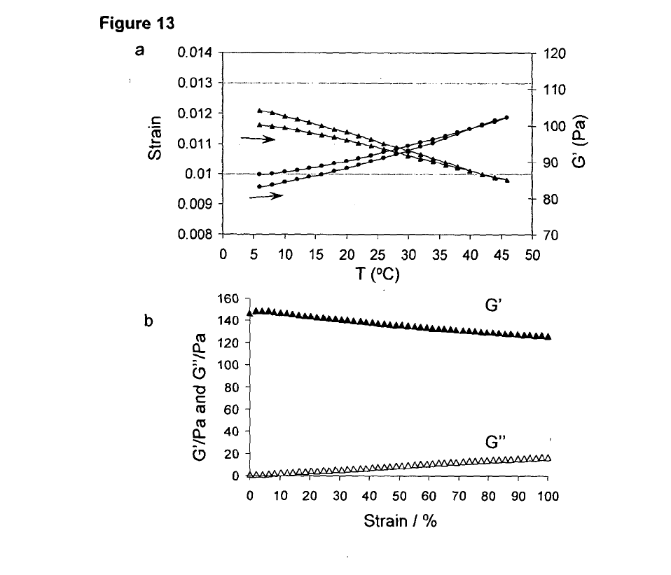

FIG. 13: (a) Temperature dependence of strain and storage modulus (G′) during heating and cooling of hydrogel samples at 2° C./min with 2 minute equilibration time. Arrows show the start of the temperature sweeping. The results showed that the strain of the hydrogel underwent shrinkage for about 11% at 1N when the temperature increased from 10° C. to 22° C. corresponding to a larger storage modulus (G′) at higher temperature. (b) The mechanical properties of HG21 showing the results of storage modulus (G′) and loss modulus (G″) versus strain (%).

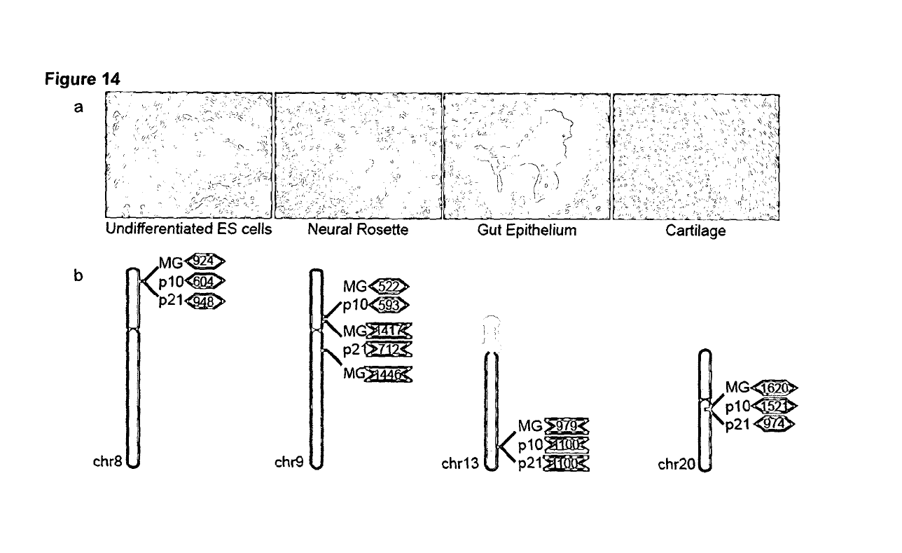

FIG. 14:

Characterisation of RH1 hESCs Cultured on Thermomodulatable HG21.

(a) Kidney capsule teratomas, induced by injection of RH1 cells cultured for 20 passages on HG21 into NOD/SCID mice, contain tissue derived from all three primary germ layers. From left to right: islands of undifferentiated EC/ES cells, neural rosettes (ectoderm), gut epithelium (endoderm), and cartilage (mesoderm). Sections were stained with Masson's trichrome. (b) Schematic summary of copy number variations detected by comparative genome hybridisation analysis of RH1 cells using a Nimblegen™ 135K probe array following culture on HG21 for 10 and 21 passages (p10, p21) and contemporaneously age matched Matrigel™ (MG) for 21 passages. Depicted are micro deletions and duplications and their size in kilobase pairs (>#<, <#>, respectively) in relation to their location on chromosomes for each genomic DNA sample. No gross chromosomal aneuploidies were detected between HG21 and MG grown cells. Copy number variations occurring in the former also occurred in the latter on chromosomes 8, 9, 13 and 20.

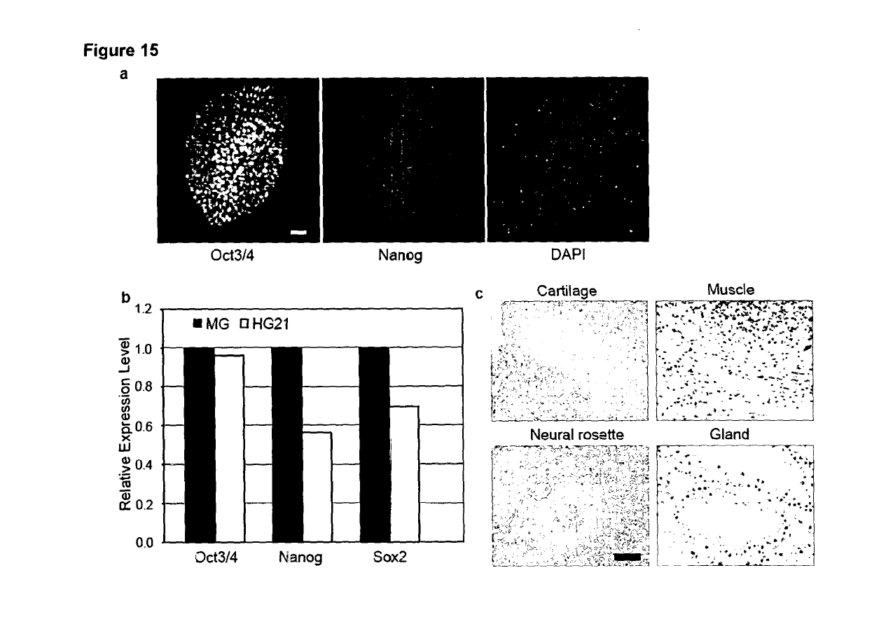

FIG. 15. Characterisation of H9 cells cultured long term on HG21.a, H9 cells cultured on HG21-coated cover slips for 6 passages retain expression of transcription factors Oct3/4 and Nanog as assessed by immunocytochemistry.b, H9 cells cultured on HG21-coated coverslips for 6 passages retain RNA expression of transcription factors Oct3/4, Nanog and Sox2. RNA levels are expressed as fold change compared with the expression of respective genes in H9 cells grown on Matrigel (MG) for 6 passages c, Hematoxylin & eosin staining of sections of teratomas formed in testes of fox chase SCID beige mice following injection of H9 cells cultured on HG21 for 9 passages shows that the teratomas contain derivatives of all three germ layers: Mesoderm (cartilage and muscle), ectoderm (neural rosette) and endoderm (glandular structures).

### EXAMPLES

1. Materials

All chemicals used for hydrogel microarray fabrication were purchased from Sigma-Aldrich except tridecafluoro-1,1,2,2-tetrahydrooctyl-dimethylchlorosilane (FDS) (ABCR GmbH Co, KG). N-acryloyl-N′-propylpiperazine (NANPPA), 2,2′-(ethylenedioxy)bis(ethylamine) mono-acylamide (EOA) was synthesized in house while N-isopropylacrylamide (NIPAA) was purified by recrystallization as described previously.^{17,20} Other chemicals were used as received.

2. Methods

2.1 Fabrication of Polymer Hydrogel Microarrys

Tertiary polymer blend hydrogel microarrays (28×87 spots) were fabricated using inkjet printing. Initially masked glass slides were generated by printing sucrose solution (20% wt) on cleaned glass slides and followed by treatment with FDS for over 4 h followed by rinsing with acetone and water to remove excess FDS and sucrose. Slides were treated with 3-trimethoxysilane propylmethacrylate overnight before rinsing with acetone. For the array fabrication, the redox initiators ammonium persulfate (APS) and TEMED were used as previously reported.¹⁷ In the printing/fabrication procedure, an aqueous solution of the crosslinker 2,2′-methlenebisacrylamide (MBA, 3% wt/wt) was added to the solutions of the 18 main monomers (see Table 1) before printing, with each combination of two monomers printed in quadruplicate. The monomer ratios varied in steps of 14% (FIG. 4). Initially, 6 drops of the initiator (APS, 1% wt/wt in distilled water) were printed. These were overprinted with the two monomers (16 drops in total) and finally 6 drops of TEMED (28.5% wt/wt aqueous solution pH 7) at 20±2° C. with the humidity in the printing chamber controlled at 70±5%. In total 87 lines were printed giving 609 different polymers (in quadruplicate=2436 polymer spots). The printed slides were kept at 37° C. for 8 h before gently rinsing with water and ethanol. The slides were UV sterilised for 20 minutes and washed with PBS (x2) prior to cell culture. The same procedure was used for the preparation of the “hit” hydrogel microarrays.

TABLE 1
Monomers used for preparing the polymer hydrogel microarrays.

| | | | Con. in H₂O |
|---|---|---|---|
| Monomers | | Structure | (% wt)^{a)} |
| 1 | MBA | 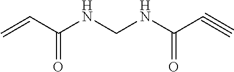 | 0.7 |
| 2 | NIPAA | 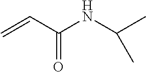 | 14 |
| 3 | DMOBAA | 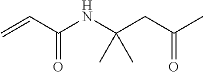 | 14 |
| 4 | NANPPA | 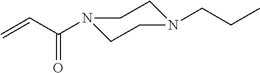 | 14 (pH7) |
| 5 | DEAA | 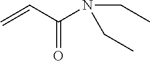 | 2.8 |
| 6 | DMAEMA | 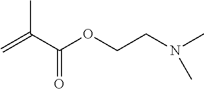 | 14 (pH7) |
| 7 | DEAEA | 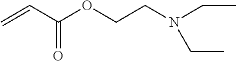 | 14 (pH7) |
| 8 | DMAA | 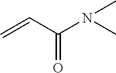 | 14 |
| 9 | AAm | 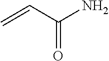 | 14 |
| 10 | EOA | 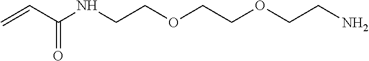 | 7 |
| 11 | AEtMA-Cl | 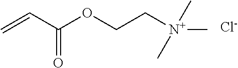 | 14 |
| 12 | HBA | 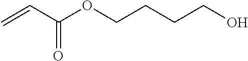 | 7 |
| 13 | HEMA | 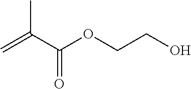 | 7 |
| 14 | HPMA | 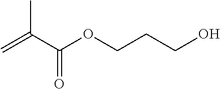 | 7 |
| 15 | PEG6MA | 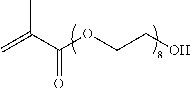 | 14 |
| 16 | AAH | 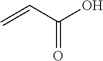 | 10 (pH7) |
| 17 | CEA | 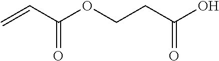 | 12 (pH7) |
| 18 | CEAO | 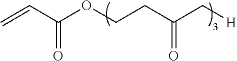 | 14 (pH7) |
| 19 | PEMA | 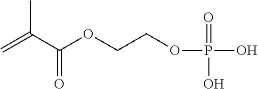 | 7 (pH6) |

Notes:
^{a)}Monomers were dissolved in water with the pH adjusted to pH 7 using NaOH (2N) or HCl (2N). The pH of the CEAO solution was adjusted using a Na₂CO₃ (20% wt) solution.

2.2 Hydrogel Coated Coverslips

Hydrogel coated cover slips and slides were prepared as described previously.^{Errort Bookmark not defined.} Briefly, solutions of the monomers, crosslinker and photoinitiator in N-methyl-2-pyrrolidone were coated on treated coverslips and exposed to 365 nm UV light for 30 minutes and placed in a 50° C. oven overnight. The polymer coated coverslips were then washed with ethanol, acetone and dried in air.

2.3 Human Embryonic Stem Cell (hESC) Culture

RCM-1, RH1 and H9 hES cell lines were used for the high-content screens. The cells were cultured on plastic tissue culture well plates (Corning inc. NY) coated with Matrigel (BD) in a feeder-independent environment with chemically defined mTESR1 medium (StemCell Technologies) at 37° C., 5% CO₂. Cells were passaged using a collagenaseIV (Invitrogen)/scraping regime (or thermally—see below).

2.4 Human ESC culture on Polymer Hydrogel Microarrys

Human ESC were seeded at a density of 60,000 cells/cm² on microarray slides in 5 ml mTeSR1 containing 1% penicillin/streptomycin (Invitrogen), 1% fungizone (Invitrogen) and 10 μM ROCK inhibitor (Calbiochem). Cells were incubated for 2 days before fixing and staining with DAPI.

To optimise the evaluation of the cell number analysis on hydrogel spots, we introduced a 4 level cell counting system, which has the benefit in the initial screening of identifying possible polymers. Level 0 was used if less than 10% of the area of a spot was covered with cells; level 1, 10% to 40%; level 2, 40% to 70% and level, 3 70% to 100%. Combining the results from two independent screens, 120 ‘hit’ polymers were chosen because of their high levels of binding (Table 2).

TABLE 2
Data analysis* of the initial hESC (RCM1) screening on the polymer hydrogel
microarrays.

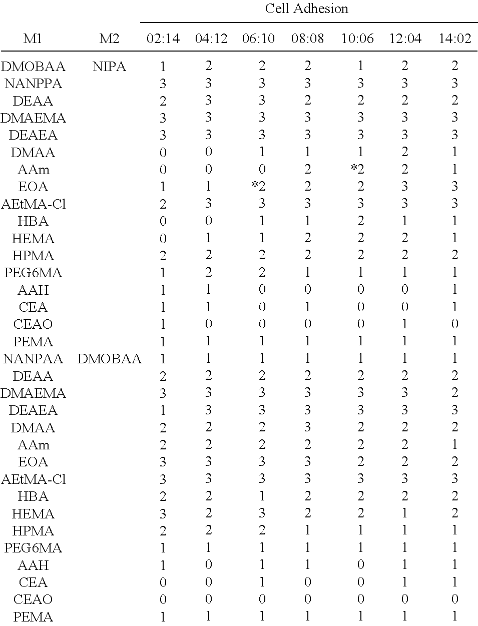

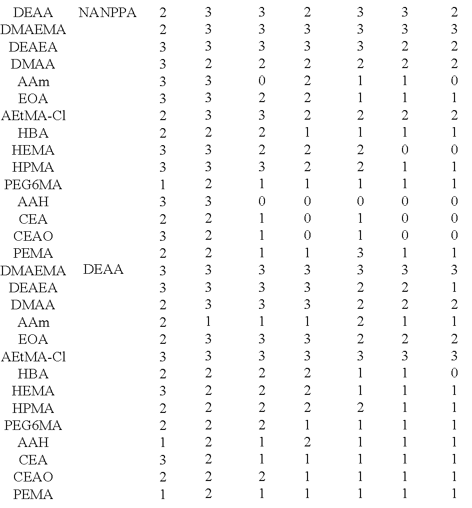

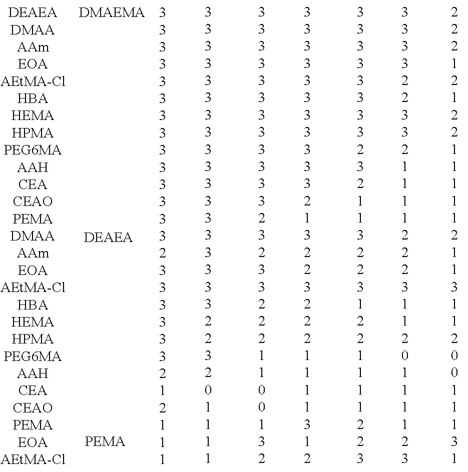

Key: 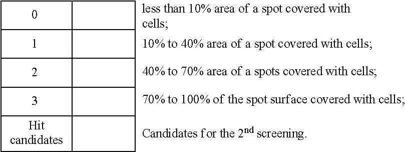 [SMILES: C1C2CC3C4C5C6CC7CC8C9C%10C1C23C%104C95C786]
*Data are from two independent arrays.

Some monomer combinations showed very good cellular affinity in all ratios such as NANPPA/NIPA. In this case, three ratios (4/12, 8/8 and 12/4) were arbitrarily chosen as hit polymers.

The ‘hit’ microarrays were printed with 9 replicates per polymers and incubated with RCM1 for 2, 4 and 7 days respectively. After fixation the arrays were stained for OCT4 and Nanog and treated with DAPI before scanning. To count the bound cells per unit area on hydrogel spots, the scanned images were analysed automatically using Pathfinder software and then corrected manually. The Top 50 hydrogels are presented in FIG. 5, showing that the cells proliferated on the polymer spots. Some hydrogels showed most cells on the 4^{th} day which could mean detachment of cells (maybe because colonies were washed away during medium refreshment as they become too over-crowded). Most RCM1 cells on polymer spots expressed OCT4/Nanog after 7-day culture (FIG. 6a). Some small stem cell colonies could be observed on hydrogel spots as shown in FIG. 6b. The top polymers were scaled up for analysis of longer-term culture.

2.4 hESC Culture on Polymer Coated Glass Surface

Hydrogel-coated coverslips were sterilised for 20 min under UV light and were then washed with Dulbeccos phosphate buffered saline (PBS) (Sigma), before cells were seeded at a density of 90 000 cells/cm². Once 80% confluency was reached, cells were split at a suitable ratio (1:1.5 or 1:2) by thermodetachment at 15±3° C. for 30 minutes; they were then collected, pelleted, resuspended in mTESR1 and replated accordingly. Cells were maintained by daily replacing ⅔ of the media with fresh mTESR1.

In this study the top 35 hydrogels were coated on glass coverslips (13 mm) as described earlier (Table 3).

TABLE 3
Top 35 candidates chosen from the results of the hit-
array screening with bFGF complemented mTeSR1.

| Polymer | Printing drop ratios |
|---|---|
| Ref. No.* | mon1/mon2 |
| 60 | AEtMA-Cl/DEAA = 4/12 |
| 61 | AEtMA-Cl/DEAA = 8/8 |
| 62 | AEtMA-Cl/DEAA = 12/4 |
| 69 | DMAEMA/DEAA = 4/12 |
| 70 | DMAEMA/DEAA = 8/8 |
| 71 | DMAEMA/DEAA = 12/4 |
| 51 | DEAEA/DMAEMA = 4/12 |
| 52 | DEAEA/DMAEMA = 8/8 |
| 53 | DEAEA/DMAEMA = 12/4 |
| 28 | AEtMA-Cl/DMAEMA = 4/12 |
| 29 | AEtMA-Cl/DMAEMA = 8/8 |
| 26 | AAH/DMAEMA = 4/12 |
| 45 | AAm/DMAEMA = 4/12 |
| 46 | AAm/DMAEMA = 8/8 |
| 48 | DMAA/DMAEMA = 4/12 |
| 49 | DMAA/DMAEMA = 8/8 |
| 115 | DMAEMA/NIPA = 4/12 |
| 116 | DMAEMA/NIPA = 8/8 |
| 117 | DMAEMA/NIPA = 12/4 |
| 110 | AEtMA-Cl/NIPA = 8/8 |
| 84 | DMAEMA/NANPPA = 8/8 |
| 85 | DMAEMA/NANPPA = 12/4 |
| 101 | DMAEMA/DMOBAA = 8/8 |
| 102 | DMAEMA/DMOBAA = 12/4 |
| 19 | AEtMA-Cl/DEAEA = 4/12 |
| 20 | AEtMA-Cl/DEAEA = 8/8 |
| 21 | AEtMA-Cl/DEAEA = 12/4 |
| 7 | AAH/DEAEA = 2/14 |
| 9 | HPMA/DEAEA = 4/12 |
| 10 | HEMA/DEAEA = 2/14 |
| 13 | EOA/DEAEA = 2/14 |
| 15 | AAm/DEAEA = 2/14 |
| 16 | DMAA/DEAEA = 8/8 |
| 17 | DMAA/DEAEA = 4/12 |
| 5 | DMAEMA/PEMA = 12/4 |

After 7 day culture in mTeSR1, hydrogels were judged by the number of adherent cells and their morphology. It was observed that cells on some hydrogels became >75% confluent with good morphology colonies. After low temperature treatment, most of the cells were detached and could be broken down to smaller cellular aggregates by gentle pipetting. Then the cells were plated onto fresh polymer hydrogel coverslips. During the second passage, 10 hydrogels which showed the best colony morphology, easiest thermo-detachment and high cellular viability were chosen for scale-up (FIG. 7).

2.5 Differentiation

2.5.1 Hepatocyte Differentiation

Cells were split at 1:1.5 ratio onto hydrogel coated coverslips by thermodetachment. Once 25-30% confluency was reached the cells were cultured with 1 ml of RPMI 1640 (Invitrogen) supplemented with 2% B27 (Invitrogen)+100 ng/ml Activin A (Peprotech) and 50 ng/ml Wnt3a (Peprotech) for 3 d. Media was changed every 24 hours. Cells were subsequently treated with DMSO media consisting of KO-DMEM (Invitrogen), 20% knock out serum replacement (Invitrogen), 1% non essential amino acids (Invitrogen) , 0.5% L-glutamine (Invitrogen), 0.2% β-mercaptoethanol (Invitrogen) and 1% dimethylsulphoxide (DMSO) (Sigma) every other day for 5 d. Cells were then fed L-15 media consisting of Leibovitz L15 Media modified (Sigma), 10% Tryptose phosphate broth (Sigma), 10% Foetal Calf Serum (Gibco); 1 μM Insulin (bovine pancreas) (Sigma), 10 μM hydrocortisone hemisuccinate (Sigma), 1.2% L-Glutamine (Gibco) and 50 μg/ml Ascorbic Acid (Sigma). This was supplemented with 10 ng/ml hepatocyte growth factor (Peprotech) and 20 ng/ml Oncostatin M (R&D systems) every other day for 9 days. After 17 d of differentiation the cells were collected for analysis.

2.5.2. Neuronal Differentiation

RCM-1 cells grown on hydrogels for more than 20 passages were neuralised using a dual SMAD inhibition approach as previously described. At 70% confluency they were provided with CDM medium; 50% IMDM (Invitrogen), 50% F12 (Invitrogen), 5 mg/ml BSA, 1% Lipid 100× (Invitrogen), 0.09% Monothioglycerol (Sigma), 10 mg/ml Insulin (Roche) and 30 mg/ml Transferin (Roche) supplemented with 0.2 μM Dorsomorphin (Calbiochem), 1 μM Activin inhibitor (Calbiochem) and 0.1 mM N-acetyl cystein (Sigma), which was subsequently changed every 2 days.

After 7 days the cells were thermodetached and colonies harvested and resuspended in fresh CDM supplemented initially with 5 ng/ml bFGF. After neuroectoderm had been firmly established the concentration of bFGF was increased to 20 ng/ml and 20 ng/ml EGF was added to the medium. The emerging spheres were maintained for 2 months by passaging weekly: a uniform suspension of neural stem cell clusters (neurospheres) was obtained by roughly dissociating spheres with a razor blade and split 1:2. Neurospheres were then plated on Matrigel (reduced growth factor, B&D) in Neurobasal (Invitrogen), supplemented with 0.04% B27 (Invitrogen), 1% L-glutamine (Invitrogen), 0.01% NEAA (Invitrogen), 1% Penicillin/Streptomycin (Invitrogen) and 1% Fungizone (Invitrogen). For the first 48 hours cells were cultured in the presence or absence of gamma secretase inhibitor (Calbiochem) and grown for a further 12 days before processing.

2.5.3. Osteogenic Differentiation

RCM1 cells grown on polymer for more than 20 passages were thermally detached and plated onto polymer coated coverslips. hES-MPs were used as positive controls. To each of the treatment wells 0.5 ml of STEMPRO osteogenesis differentiation medium (Invitrogen A10072-01) was prepared and added in accordance with the manufacturers instructions. Untreated controls were maintained in standard media (mTESR and hES-MP medium). Cells were fixed in 4% PFA after 21 days of treatment and osteogenic differentiation was determined with von Kossa staining.

2.6 Embryoid Body (EB) Formation and Differentiation

hES colonies were detached and dissociated by pipetting until a uniform suspension of cell aggregates was obtained. Cells were then plated in low cluster plates (Corning) and grown in EB medium; DMEM (Invitrogen), 20% Foetal calf serum (Sigma), 1% L-Glutamine (Invitrogen), 1% NEAA (Invitrogen), 0.2% 2-mercaptoethanol (Invitrogen) for 7 days as a suspension culture. Subsequently, EBs were harvested and incubated with PBS-based enzyme-free cell dissociation solution (Sigma) for 10 minutes at 37° C. Dissociated EBs were then plated on Matrigel at 30% confluency in the presence of 10 μM Y-27632 and grown for another 7 days in EB differentiation medium; Advanced RPMI-1640 (Invitrogen), B27 (Invitrogen), penicillin (Invitrogen) and streptomycin (Invitrogen) in the presence of differentiation factors: Mesoderm was obtained by adding 100 ng/ml Activin A (R&D systems) for 1 day followed by 6 days of treatment with 10 ng/ml BMP-4 at (R&D systems); for endoderm the cells were treated with EB differentiation medium supplemented with 100 ng/mlActivin-A for 6 days; ectoderm was obtained by incubating the cells 3 μg/ml retinoic acid for 7 days. The present inventors have also shown that the hES cells are capable of forming teratomas following injection under the kidney capsule of immunodeficient mice, which supports the fact that pluripotency of the cells is maintained. See FIGS. 14 and 15. FIG. 14 also provides data showing karyotype maintenance.

2.8 Flow Cytometry Analysis

Cells were dissociated by incubation with 0.025% trypsin/EDTA for 5-10 minutes at 37% degrees, resuspended in FACS PBS (PBS+0.1% BSA+0.1% sodium Azide) and incubated with preconjugated antibodies (SSEA-4, FITC; SSEA-1, Phycoerythrin (PE); biotinylated Tra1-60 (all Biolegend) in FACS PBS) for 20-40 min. Tra1-60 biotin detection was performed with streptavidin-conjugated Allophycocyanine (APC). Data were obtained using flow cytometry (Calibur) and analysed using Flow Jo.

TABLE 4
Flow cytometry analysis of hESCs cells in mTeSR1 medium for
SSEA-1 (stem cell early differentiation marker) and stem
cell membrane markers of pluripotency: SSEA-4 (glycolipid
antigens) and TRA-1-60 (a keratan sulfate antigen). The percentage
of positive cells is listed in parentheses.

| | SSEA-1 | SSEA-4 | TRA-1-60 |
|---|---|---|---|
| RCM1 HG9 p20 | 3% | 52% | 32% |
| RCM1 HG21 p20 | 4% | 65% | 33% |
| RCM1 p78 | 1% | 91% | 77% |
| H9 HG9 p5 | 1% | 14% | 65% |
| H9 HG19 p5 | 1% | 13% | 71% |
| H9 HG20 p5 | 1% | 12% | 74% |
| H9 HG21 p5 | 3% | — | — |
| H9 p73 | 1% | 15% | 75% |
| RH1 HG20 p11 | 7% | 61% | 61% |
| RH1 HG21 p11 | 2% | 80% | 66% |

2.9 Immunostaining of hESCs on Coverslips

Cells were stained using a standard immunocytochemistry protocol. Briefly, hES cells on hydrogels were washed once with PBS and fixed with 4% PFA. After permeabilisation in 0.2% Igepal (Sigma) and blocking in 10% normal rabbit serum (Millipore), cells were incubated with primary antibodies OCT-4 (Santa Cruz Biotechnology), Nanog (R&D systems), GFAP (DACO), β3-tubulin (Sigma), Nestin (Millipore), AFP (Sigma), Albumin (Sigma) for 2-4 hours at RT or overnight at 4° C. Visualisation with secondary antibodies was performed using Alexafluor antibodies 488, 555, 568, and 647 (Invitrogen), and nuclei were labelled using 4′,6-diamidino-2-phenylindole, dilactate (DAPI). Imaging analysis was carried out using a Leica SPE microscope.

2.10 DNA Extraction

DNA was extracted using a standard phenol/chloroform precipitation method. Briefly, pellets of hES cells were incubated O/N in proteinase K and lysis buffer. DNA was precipitated with phenol and chloroform, washed with 5M ammonium acetate, isopropanol and 80% ethanol and the DNA was rehydrated in H₂O and stored at −20° C.

2.11 Karyotype Analysis

Comparative genome hybridisation was performed and analysed at the Cytogenetics unit at the Western General Hospital, Edinburgh, UK by a standardised comparative genome hybridisation method using the NimbleGen Nimblegen 135 k array (v. 3) (Table 5).

TABLE 5
Summary of the comparative genomic hybridization (CGH) * analysis of RH1
and H9 hESCs after long-term culture on HG21 and Matrigel respectively.

| | | | | Size | Data | | | |
|---|---|---|---|---|---|---|---|---|
| hESCs | Chromosome | Start | End | (Kb) | Points | Genes | Results | Cytoband |
| RH1-MG-P91^{a} | chr20 | 29,341,653 | 30,961,136 | 1620 | 66 | 35 | Duplication | q11.21 |
| (Control) | chr13 | 98,032,054 | 99,010,744 | 979 | 77 | 7 | Deletion | q32.2, q32.3 |
| | chr9 | 68,496,345 | 69,942,480 | 1446 | 13 | 8 | Deletion | |
| | chr9 | 45,284,971 | 46,702,333 | 1417 | 18 | 4 | deletion | |
| | chr9 | 44,667,132 | 45,188,993 | 522 | 8 | 1 | Duplication | |
| | chr8 | 7,240,496 | 8,164,912 | 924 | 42 | 23 | Duplication | |
| RH1-HG21-P10 | chr20 | 29,306,527 | 30,827,881 | 1521 | 63 | 34 | Duplication | q11.21 |
| | chr13 | 97,910,519 | 99,010,744 | 1100 | 86 | 8 | Deletion | q32.2, q32.3 |
| | chr9 | 44,595,704 | 45,188,993 | 593 | 9 | 1 | Duplication | |
| | chr8 | 7,276,315 | 7,880,533 | 604 | 37 | 17 | Duplication | |
| RH1-HG21-P21 | chr20 | 29,306,527 | 30,280,717 | 974 | 48 | 28 | Duplication | q11.21 |
| | chr13 | 97,910,519 | 99,010,744 | 1100 | 86 | 8 | Deletion | q32.2, q32.3 |
| | chr9 | 45,284,971 | 45,997,390 | 712 | 7 | 2 | Deletion | |
| | chr8 | 7,217,188 | 8,164,912 | 948 | 44 | 24 | Duplication | |
| H9-MG-P73 | chr20 | 29,401,318 | 29,951,666 | 550 | 20 | 16 | Duplication | q11.21 |
| (Control) | chr14 | 64,939,983 | 65,442,211 | 502 | 19 | 2 | Duplication | q23.3 |
| H9-HG21-P9 | chr20^{b} | 29,401,318 | 29,951,666 | | | | Duplication | q11.21 |
| | chr16 | 32,000,280 | 33,365,421 | 1365 | 14 | 6 | Deletion | |

* Microarray analysis using a Nimblegen 135K whole genome oligonucleotide array (HG18, mean probe spacing 12,524 bp).
^{a}RH1-MG-P91 means hESC RH1 cells culture on MG matrix for 91 passages before the CGH analysis.
^{b}The array analysis displayed the 20q11.21 duplication as series of contiguous duplications (which represent a single 550 kb duplication overall), the duplication is not listed automatically on the summary by the Nimblegn software.

2.12 Growth Rate Analysis

To obtain population doubling rates of hES cells on hydrogels, cells were counted at each passage using a haemocytometer. Triplicate counts were gathered at each passage and viability was assesed by staining with trypan blue (Sigma).

2.13 Rheology Analysis

Rheology analysis was carried out using a TA instrument (AR-2000, 40 mm 4° steel cone) with an oscillation frequency of 1 Hz and oscillation stress of 1 Pa. Hydrogel samples were washed in PBS and refreshed every 24 hours for one week before cutting to discs with a ˜3 mm thick and ˜2 cm in diameter.

2.14 X-ray Photoelectron Spectroscopy (XPS) Analysis

XPS analysis was carried out on a Thermo VG Scientific Sigma Probe, with an Al k_{α} X-ray source, a pass energy of 80 eV and 0.5 eV steps (scanning −10.00 to 600.00 eV).

Although the suitability of thermoresponsive polymers for mouse embryonic stem cell culture^{17,21}, has been reported previously, Loh et al observed reduced growth rates when culturing mouse embryonic stem cells on thermoresponsive co-polymers compared to gelatin controls²¹. Although they demonstrated that these gelatin-containing polymers support mouse embryonic stem cell growth and cell release at very low temperature (4° C.), cell cultures lasted only 3-4 days, without cell passaging. To our knowledge, the work presented herein is the first to report long term culture and gentle serial passaging of human embryonic stem cells using a thermomodulatable synthetic polymer, verified with independent cell lines. The fact that cells maintained an undifferentiated state, did not acquire harmful genetic aberrations, and retained pluripotency strongly suggest that the polymers of the present invention are promising candidate substrates for use in large scale, GMP-compliant pluripotent cell growth, such as hES production for clinical purposes.

### REFERENCES

¹ Vazin, T. & Freed, W. J. Human embryonic stem cells: Derivation, culture and differentiation: A review. Restor. Neurol. Neurosci. 28, 589-603 (2010).
² Lei, T. et al. Xeno-free derivation and culture of human embryonic stem cells: current status, problems and challenges. Cell Res. 17, 682-688 (2007).
³ Hakala, H. et al. Comparison of Biomaterials and Extracellular Matrices as a Culture Platform for Multiple, Independently Derived Human Embryonic Stem Cell Lines. Tissue Eng. Part A 15, 1775-1785 (2009).
⁴ Ludwig, T. E. et al. Derivation of human embryonic stem cells in defined conditions. Nat. Biotech. 24, 185-187 (2006).
⁵ Brafman, D. A. et al. Defining Long-Term Maintenance Conditions of Human Embryonic Stem Cells With Arrayed Cellular Microenvironment Technology. Stem Cells Dev. 18, 1141-1154 (2009).
⁶ Prowse, A. B. Wilson, J. Osborne, G. W. Gray, P. P. & Wolvetang, E. J. Multiplexed staining of live human embryonic stem cells for flow cytometric analysis of pluripotency markers. Stem Cells Dev. 18, 1135-1140 (2009).
⁷ Couture, L. A. Scalable pluripotent stem cell culture. Nat. Biotech. 28, 562-563 (2010).
⁸ Singh, H., Mok, P., Balakrishnan, T., Rahmat, S. N. B. & Zweigerdt, R. Up-scaling single cell-inoculated suspension culture of human embryonic stem cells. Stem Cell Res. 4, 165-179 (2010).
⁹ Oh, S. K. W. et al. Long-term microcarrier suspension cultures of human embryonic stem cells. Stem Cell Res. 2, 219-230 (2009).
¹⁰ Rodin, S., et al. Long-term self-renewal of human pluripotent stem cells on human recombinant laminin-511, Nature Biotech. 28, 611-617 (2010).
¹¹ Noaksson, K. et al. Monitoring differentiation of human embryonic stem cells using real-time PCR, Stem Cells 23, 1460-1467 (2005).
¹² Braam, S. R. et al. A Sonneberg and C L Mummery, Recombinant vitronectin is a functionally defined substrate that supports human embryonic stem cell self-renewal via alphavbeta5 integrin, Stem Cells, 26, 2257-2265 (2008).
¹³ Li, Y. J., Chung, E. H., Rodriguez, R. T., Firpo, M. T. & Healy, K. E. Hydrogels as artificial matrices for human embryonic stem cell self-renewal. J. Biomed. Mater. Res. Part A 79, 1-5 (2006).
¹⁴ Villa-Diaz, L. G. et al. Synthetic polymer coatings for long-term growth of human embryonic stem cells. Nature Biotechnol. 28, 581- 583 (2010).
¹⁵ Melkoumian, Z. et al. Synthetic peptide-acrylate surfaces for long-term self-renewal and cardiomyocyte differentiation of human embryonic stem cells. Nature Biotechnol. 28, 606-610 (2010).
¹⁶ Mei, Y. et al. Combinatorial development of biomaterials for clonal growth of human pluripotent stem cells. Nat. Mater. 9, 768-778 (2010).
¹⁷ Zhang, R., Liberski, A., Sanchez-Martin, R. & Bradley, M. Microarrays of over 2000 Hydrogels—Identification of substrates for cellular trapping and thermally triggered release. Biomaterials 30, 6193-6201 (2009).
¹⁸ Caisander, G., et al. Chromosomal integrity maintained in five human embryonic stem cell lines after prolonged in vitro culture. Chromosome Res. 14, 131-137 (2006).
¹⁹ Stephenson, E., et al. Safety paradigm: genetic evaluation of therapeutic grade human embryonic stem cells. J. R. Soc. Interface 7, S677-S688 (2010).
²⁰ Zhang, R., Liberski, A., Khan, F., Diaz-Mochon, J. J. & Bradley, M. Inkjet fabrication of hydrogel microarrays using in situ nanolitre-scale polymerisation. Chem. Commun. 11, 1317-1319 (2008).
²¹ Loh, X. J. et al. Surface coating with a thermoresponsive copolymer for the culture and non-enzymatic recovery of mouse embryonic stem cells. Macromolecular bioscience 9, 1069-1079 (2009).

1-27. (canceled)

28. A method of releasing stem cells from a substrate, comprising:
a) providing stem cells adhered on a substrate, wherein the substrate comprises a polymer hydrogel wherein the polymer hydrogel comprises a random structure according to the following composition:
Xn; Yn′; Zn″,
wherein X and Y represent first and second monomers and Z represents a cross-linking agent;
wherein X may be selected from the group consisting of hydroxypropyl methacrylate (HPMA), [2-(acryloyloxy)ethyl]trimethylammonium chloride (AEtMA-Cl), and 2-(dimethylamino)ethyl methacrylate (DMAEMA);
wherein Y may be selected from the group consisting of 2-(diethylamino)ethyl acrylate (DEAEA), N-(1,1-dimethyl-3-oxobutyl)acrylamide (DMOBAA), N-isopropylacrylamide (NIPAA), and N,N-diethylacrylamide (DEAA);
wherein Z may be selected from the group consisting of N,N′-methylenebisacrylamide (MBA), ethylene glycol diacrylate, tetra(ethylene glycol) diacrylate, glycerol dimethacrylate, poly(ethylene glycol) diacrylate and (O,O-Bis(3-aminopropyl)polyethylene glycol) diacrylamide;
wherein n and n′ represent the molar ratio in percent of monomers X and Y relative to each other ranging between approximately 10%-90% and 90-10%, respectively; and
wherein n″ represents the molar ratio in percent of Z relative to X and Y combined ranging between approximately 1%-20%; and
b) releasing the stem cells from the substrate with a thermal treatment.

29. The method according to claim 28, wherein said stem cells are cultured on the substrate at an initial temperature, and the thermal treatment is a lower temperature than the initial temperature.

30. The method or device/apparatus according to claim 29, wherein the initial temperature ranges between approximately 30° C. to 39° C. and the thermal treatment temperature ranges between approximately 5° C. to 29° C.

31. The method according to claim 28, wherein the thermal treatment ranges between approximately 10 minutes to 1 hour.

32. The method according to claim 29, wherein the initial temperature ranges between approximately 35° C. to 39° C., and the thermal treatment ranges between between approximately 12° C. to 17° C., for between approximately 15 minutes to 45 minutes.

33. The method according to claim 28, wherein n and n′ range between approximately 20% to 80% and 80% to 20%, respectively.

34. The method according to claim 28, wherein n″ ranges between approximately 2% to 10%.

35. The method according to claim 28, wherein X is HPMA and Y is DEAEA.

36. The method according to claim 28, wherein X is AetMA-Cl and Y is DEAEA.

37. The method according to claim 28, wherein the polymer comprises AetMA-Cln, DEAEAn′, MBAn″, wherein n and n′ ranges between approximatly 30% to 70% and 70% to 30%, respectively.

38. The method according to claim 28, wherein the polymer comprises HPMAn, DEAEAn′, MBAn″, wherein n and n′ ranges between approximately 30% to 70% and 70% to 30%, respectively.

39. The method according to claim 28, wherein the stem cells are selected from the group consisting of adult stem cells, embryonic stem cells, pluripotent stem cells, and induced pluripotent stem cells (iPS).

40. The method according to claim 28, wherein the stem cells are selected from the group consisting of mammalian stem cells, human stem cells, primate stem cells, ungulate stem cells, ruminant stem cells, and rodent stem cells.

41. The method according to claim 28, wherein the stem cell is a mesenchymal stem cell.

42. A polymer hydrogel which comprises a random structure according to the following composition:
Xₙ; Y_{n′}; Z_{n″},
wherein X and Y represent first and second monomers and Z represents a cross-linking agent;
wherein X may be selected from the group consisting of hydroxypropyl methacrylate (HPMA), [2-(acryloyloxy)ethyl]trimethylammonium chloride (AEtMA-Cl), 2-(dimethylamino)ethyl methacrylate (DMAEMA);
wherein Y may be selected from the group consisting of 2-(diethylamino)ethyl acrylate (DEAEA), N-(1,1-dimethyl-3-oxobutyl)acrylamide (DMOBAA), N-isopropylacrylamide (NIPAA), and N,N-diethylacrylamide (DEAA);
wherein Z may be selected from the group consisting of N,N′-methylenebisacrylamide (MBA) ethylene glycol diacrylate, tetra(ethylene glycol) diacrylate, glycerol dimethacrylate, poly(ethylene glycol) diacrylate and (O,O-Bis(3-aminopropyl)polyethylene glycol) diacrylamide;
wherein n and n′ represent the molar ratio in percent of monomers X and Y relative to each other and range between approximately 10% to 90% and 90 to 10%, respectively;
wherein n″ represents the molar ratio in percent of Z relative to X and Y combined and ranges between approximately 1% to 20%.

43. The polymer hydrogel according to claim 43, wherein n and n′ range between approxiamtely 20% to 80% and 80% to 20%, respectively.

44. The polymer hyrdogel according to claim 43, wherein n″ ranges beween approximately 2% to 10%.

45. The polymer hydrogel according to claim 43, wherein X is HPMA and Y is DEAEA.

46. The polymer hydrogel according to claim 43, wherein X is AetMA-Cl and Y is DEAEA.

47. A polymer hydrogel according to claim 43, comprising the following composition:
AEtMA-Clₙ; DEAEA_{n′}; MBA_{n″},
wherein n and n′ ranges between approximately 25% to 75% and 75% to 25%, respectively; and
wherein n″ ranges between approximately 4% to 8%.

48. A polymer hydrogel according to claim 43, comprising the following composition:
HPMAₙ; DEAEA_{n′}; MBA_{″},
wherein n and n′ range between approximately 25% to 75% and 75% to 25%, respectively; and
wherein n″ ranges between approximately 4% to 8%.

49. A cell culture device or apparatus for use in the culture of stem cells comprising a polymer according to claim 42.
